(19) United States
(12) Patent Application Publication
(10) Pub. No.: US 2007/0031409 A1
(43) Pub. Date: Feb. 8, 2007

(54) Recombinant DNA-molecule complex for the expression of anti-human-interferon-gamma chimeric antibodies or antibody fragments

(75) Inventors: Alfons Jozef Denis Alida Billiau, Linden Belgie (BE); Guido Frans Valentius Froyen, Herent Belgie (BE)

Correspondence Address: THE WEBB LAW FIRM, P.C., 700 KOPPERS BUILDING, 436 SEVENTH AVENUE, PITTSBURGH, PA, 15219, US

(73) Assignee: Stichting Rega VZW, Leuven Blegie (BE)

(21) Appl. No.: 11/057,064

(22) Filed: Feb. 11, 2005

Related U.S. Application Data
(63) Continuation of application No. 10/850,820, filed on May 21, 2004, now abandoned.
Application No. 10/850,820 is a continuation of application No. 10/623,905, filed on Jul. 21, 2003, now abandoned.
Application No. 10/623,905 is a division of application No. 08/286,797, filed on Aug. 5, 1994, now abandoned.
Application No. 08/286,797 is a continuation of application No. 07/766,011, filed on Sep. 26, 1991, now abandoned.

(51) Int. Cl. A61K 39/395; C12P 21/06; C12N 5/06; C07K 16/24

(52) U.S. Cl. 424/145.1; 435/69.1; 435/326; 435/320.1; 530/388.23

(57) ABSTRACT

A method for producing biologically active Fv antibody fragments that have a neutralizing effect on the anti-viral activity of human interferon-gamma (IFN-Γ) by inserting an isolated nucleic acid, having nucleotide sequences that encode the V_{H} and V_{L} domains of the D9D10 monoclonal antibody and a nucleotide sequence that encodes a linker peptide which links the V_{H} and V_{L} domains, into a suitable expression vector, in order to encode F_{V} antibody fragments. Such Fv antibody fragments can be used to treat human diseases, such as endotoxic shock, local inflammation, cerebral malaria, and autoimmune arthritis.

### CROSS REFERENCE TO RELATED APPLICATIONS

This application is a division of U.S. patent application Ser. No. 08/286,797, filed Aug. 5, 1994, which is a File Wrapper Continuation of U.S. patent application Ser. No. 07/766,011, filed Sep. 29, 1991.

### BACKGROUND OF THE INVENTION

1. Field of the Invention

The invention relates to a recombinant DNA-molecule complex for the expression of anti-human-interferon-Γ chimeric antibodies or antibody fragments.

Further, the invention relates to a method for producing anti-human-interferon-Γ (anti-HuIFN-Γ) chimeric antibodies or antibody fragments, a recombinant cell line and a recombinant E. coli-strain comprising said recombinant DNA molecule complex, anti-human-interferon-Γ chimeric antibodies, anti-human-interferon-Γ Fv-fragments, biological active preparations comprising said chimeric antibodies or Fv-fragments, the use of said biological active preparations and methods for preparing the constituents of the recombinant DNA-molecule complex.

2. Description of Related Art

Immunoglobulins (antibodies) are glycoproteins present in the blood and body fluids of higher animal species. They recognize and bind certain categories of substances that are foreign to the body (antigens) and induce effector functions that eliminate or neutralize these antigens and their carriers (viruses, bacteria, cancer cells). An immunoglobulin comprises two identical light and two also identical heavy polypeptide chains (called L- and H-chains, respectively), that are linked by means of disulfide bridges. The sequences of the N-terminal ends of the four chains are very variable and together responsible for the binding of the antigen. The variable regions, therefore, determine the specificity of the antibody. The C-terminal part of the chains has, however, a relatively constant structure and determines the nature of the effector functions, such as complement activation, antibody dependent cellular cytotoxicity and others.

Interferon-Γ (IFN-Γ) is a glycoprotein that is produced by T-cells in response to antigenic or mitogenic stimuli. In addition to its antiviral activity, interferon-Γ can also show strong immunoregulatory effects, making it an important factor in the defense mechanism against bacteria, viruses and other antigens, but also in autoimmune diseases and cancer. The contribution of IFN-Γ in these pathological reactions can, however, also be harmful for the host.

Animal model studies have clarified that monoclonal antibodies against interferon-Γ have a positive effect on endotoxic shock, local inflammation, cerebral malaria and autoimmune arthritis. The monoclonal antibodies against interferon-Γ could, therefore, possibly also be used for the treatment of related human diseases.

Because of the ongoing development of hybridoma techniques, a great number of monoclonal antibodies has become available that can be applied for the treatment of diseases. From numerous studies it is, however, found that the administration of xenogenic antibodies generates an immunoglobulin response in the recipient. The administration of human monoclonal antibodies could reduce or preclude such anti-immunoglobulin response. The production of those human monoclonal antibodies encounters ethical as well as practical objections. To avoid the above problems, it has been tried recently to strongly reduce the unwanted anti-immunoglobulin response by means of in vitro manipulation of the immunoglobulin genes, such as chimerization or humanization. Important results have already been achieved herewith.

### SUMMARY OF THE INVENTION

It has now been found that a chimeric antibody comprising, e.g., murine variable domains and human constant domains, as well as the Fv-fragment of an antibody against human interferon-Γ have an affinity for IFN-Γ and are capable of neutralizing its biological activity. It is expected that with the use of such chimeric antibodies or Fv-fragments, the anti-immunoglobulin response can be strongly reduced. This is caused by the fact that either the immunogenic murine C-regions are replaced by human C-regions or the immunogenic C-regions are removed from the antibody, while still retaining the antigen binding properties of the V-regions. The present invention provides a recombinant DNA-molecule complex for expressing a functional chimeric antibody or a functional Fv-fragment of an antibody against human interferon-Γ. The recombinant DNA-molecule complex, therefore, comprises one or more DNA-molecules comprising the suitable transcription initiation and termination signals and a DNA-sequence encoding one or both of the murine light and heavy chain variable domains of the hybridoma cell line D9D10 (Sandvig et al., Immunol. Rev. 91:51-65 (1987)) and optionally one or more human light or heavy chain constant domains.

### BRIEF DESCRIPTION OF THE DRAWINGS

FIGS. 1 and 2 show schematically the construction of plasmids encoding the chimeric L- and H-chain genes with and without an intron between the V- and C-regions;

FIG. 3 shows schematically the construction of the chimeric heavy chain with an intron (pSVgptGFH1);

FIG. 4 shows schematically the construction of the chimeric light chain with an intron (pSVhygGFL1);

FIG. 5 shows schematically the construction of the expression vector pSVgptGFH2;

FIG. 6 shows schematically the construction of the expression vector pUCHuCKnar; and

FIG. 7 shows schematically the construction of the expression vector for Fv-fragments of anti-HuIFN-1 antibody.

### DETAILED DESCRIPTION OF THE INVENTION

Preferred embodiments of the invention provide recombinant DNA-molecule complexes including two DNA-molecules. The chimeric heavy chain can either comprise a DNA-sequence encoding the V_{H}-region of hybridoma cell line D9D10, the genomic human constant Γ1-sequence of vector pBGS18-HuIgG1 (for sequence human IgG1 see: Takahashi et al. Cell 29:671-679 (1982)) downstream thereof and an intron between them, or the V_{H}-region of hybridoma cell line D9D10 and the genomic human constant domains of vector pBGS18-HuIgG1. Chimeric light chains can comprise a DNA-sequence encoding the V_{K}- region of hybridoma cell line D9D10, the genomic human C_{K}-region of vector pSVhygHuCK (Orlandi et al., Proc. Natl. Acad. Sci. USA 81:7161-7165 (1984)) downstream thereof and an intron between them or the V_{K}-region of hybridoma cell line D9D10 and the C_{K}-region of the vector pUCHuCK-Nar (Dubois, PhD-thesis (1989)). Both chimeric light chains can be combined with both chimeric heavy chains, thereby providing four possible combinations.

In another preferred embodiment of the invention, the recombinant DNA-molecule complex comprises one recombinant DNA-molecule encoding a Fv-fragment of an anti-human-interferon-Γ antibody, comprising a dicistronic DNA-sequence encoding the V_{H}-region of hybridoma cell line D9D10 and the V_{K}-region of hybridoma cell line D9D10 downstream thereof, a suitable promoter region upstream of the dicistronic DNA-sequence and a suitable terminator region downstream of the dicistronic DNA-sequence. Both V-regions might optionally be connected by means of a linker peptide. Upstream of both cistrons leader-peptide sequences are preferably comprised. The leader-peptide sequence can, for example, be the pe1B-leader peptide sequence. For detecting the Fv-fragrnent in an ELISA, a DNA-sequence encoding the TAG1-polypeptide is comprised downstream of the V_{K}-region. Between the V_{H}-region and V_{K}-region a DNA-sequence encoding a linker peptide can optionally be comprised. The sequence of the linker peptide is represented by formula Ic.

A method for producing an anti-human-interferon-Γ chimeric antibody or antibody fragment comprises the steps of:

a) preparing a recombinant DNA-molecule complex,

b) transfecting a suitable host cell with the recombinant DNA-molecule complex to obtain a recombinant host cell capable of expressing said anti-human-interferon-Γ chimeric antibody or antibody fragment; and

c) culturing said recombinant host cell so as to allow for the expression of said anti-human-interferon-Γ chimeric antibody or antibody fragment.

The present invention will be further illustrated by the following examples.

### EXAMPLE 1

Isolation and Amplification of the V_{H}- and V_{K}Fragment of Anti-HuIFN-Γ

The nucleotide sequence of both the 5′- and the 3′-ends of the V-regions of immnunoglobulins is strongly conserved. Because of that, it is possible to define oligonucleotides capable of amplifying the V_{H}- and V_{K}-regions of practically every mouse antibody (see Orlandi et al. (1989). Proc. Natl. Acad. Sci. USA 86: 3833-3837). The primers for the amplification of the V_{H}-regions are:

VH1BACK:

(SEQ ID NO: 1)

5′ AGGTSMARCTGCAGSAGTCWGG 3′
PstI
VH1FOR:

(SEQ ID NO: 2)

5′ TGAGGAGACGGTGACCGTGGTCCCTTGGCCCC 3′.
BstEII

wherein:
S = C or G
M = A or C
R = A or G
W = A or T

The primers for the amplification of V_{K}-regions are:

VK2BACK:

(SEQ ID NO: 3)

5′ GACATCGAGCTCACCCAGTCTCCA 3′
SacI
VK2FOR:

(SEQ ID NO: 4)

5′ GTTTGATCTCGAGCTTGGTGCC 3′
XhoI

By the introduction of restriction sites in the amplified DNA-fragment, the cloning of the fragments is considerably simplified.

For the amplification of 1 μg mRNA of D9D10, the first cDNA strand was synthesized in a way well known in the art. For this, 100 pmol random hexanucleotide was used as a primer because after PCR a more specific DNA-fragment was obtained therewith than when a PCR-oligonucleotide was used for the first strand synthesis. The amplification of the desired fragment was performed on the reaction product of the first strand cDNA synthesis with the two specific primers in a 50 μl reaction mixture comprising 10 mM Tris-HCl pH 8.3, 50 mM KCl, 1.5 mM MgCl₂, 0.1 mg/ml gelatine, 0.25 mM dNTP, 25 pmol per oligonucleotide primer and 2.5 U Taq-polymerase. The reaction mixture was covered with 2 drops paraffin oil and subjected to 30 to 35 cycles of 30 sec. 95° C. (denaturation), 35 sec. 61° C. (annealing) and 25 sec. 72° C. (filling-in reaction) with a last filling-in of 5 min. at 72° C. For performing the PCR, a programmable thermic block was used (PCH-2; Techne, Cambridge, GB). Electrophoresis of 5 μl of the reaction mixture on a 2% agarose gel showed the degree of purity and the yield of the amplified DNA-fragment. The other 45 μl were subjected to chloroform extraction (400 μl) after which the DNA was precipitated with NaA-c/ethanol. On this the desired reactions with the restriction enzymes were subsequently performed.

### EXAMPLE 2

Construction of Expression Vectors Encoding Chimeric Ig-Genes

1. Expression Vectors pSVgptMoVHnp and pSVhygHuCK

The expression vector pSVgptMoVHnp comprises an immunoglobulin enhanced (E), -promoter (PR) and -leader peptide (L), and a V_{H}-region from an antibody with a specificity for 4-hydroxyl-3-nitrophenacetyl (np). The unique restriction sites BamHI and HindIII allow the insertion of new sets of (PR-L-V). The vector also comprises an ampicillin resistance gene for selection in bacteria and a guanine-phosphoribosyl-transferase gene of E. coli (Ecogpt) for selection in eukaryotic cells.

The vector pSVhygHuCK is a similar expression vector wherein the human C_{K} sequence is already inserted behind the (E-VL) region. The vector comprises the selection marker hygromycin (hyg) through which eukaryotic cells after transfection become resistant against this antibiotic. Ampicillin allows selection in bacteria. The vector was a gift from Dr. Jones (Medical Research Council, Cambridge, GB).

2. General Procedure for the Construction of Chimeric Ig Genes

2.1. Constructions with an Intron between the V- and C-Regions

The promoter (PR)-, leader peptide (L)- and V_{H}-regions of the vector pSVgptMoVHnp are replaced by a DNA-sequence comprising the desired promoter and signal peptide sequences, the murine V_{H}-region of antibody D9D10 and the desired human constant domains for obtaining an expression vector encoding a chimeric heavy chain containing an intron between V and C.

The V_{L}-region of the expression vector pSVhygHuCK was replaced with a DNA-construct comprising the desired promoter and signal peptide sequences and the murine V_{K}-region of antibody D9D10 to obtain an expression vector encoding a chimeric light chain with an intron between the V_{K} and C_{K} regions.

2.2 Constructions without an Intron Between the V- and C-Regions

For the construction of expression vectors encoding a chimeric heavy chain without an intron between V and C, the PR-L-V_{H}-region of the expression vector pSVgpt-MoVHnp was replaced by a DNA-sequence comprising the desired promoter and leader peptide sequences, and a region without an intron comprising the murine V_{H}-domain together with a small part of the C_{H′}-domain of the mouse directly linked to the first human C_{H}-domain, thus obtaining an expression vector encoding a chimeric heavy chain without an intron between V and C.

For the chimeric light chain without an intron between V and C, the V_{L}-region and part of the human C_{K}-region of the expression vector pSVhygHuCK were replaced by a DNA-sequence comprising the desired promoter and leader peptide sequences and a DNA-stretch comprising the murine V_{K}-domain, part of the C_{K′}-domain of the mouse and part of the desired human C_{K}-domain.

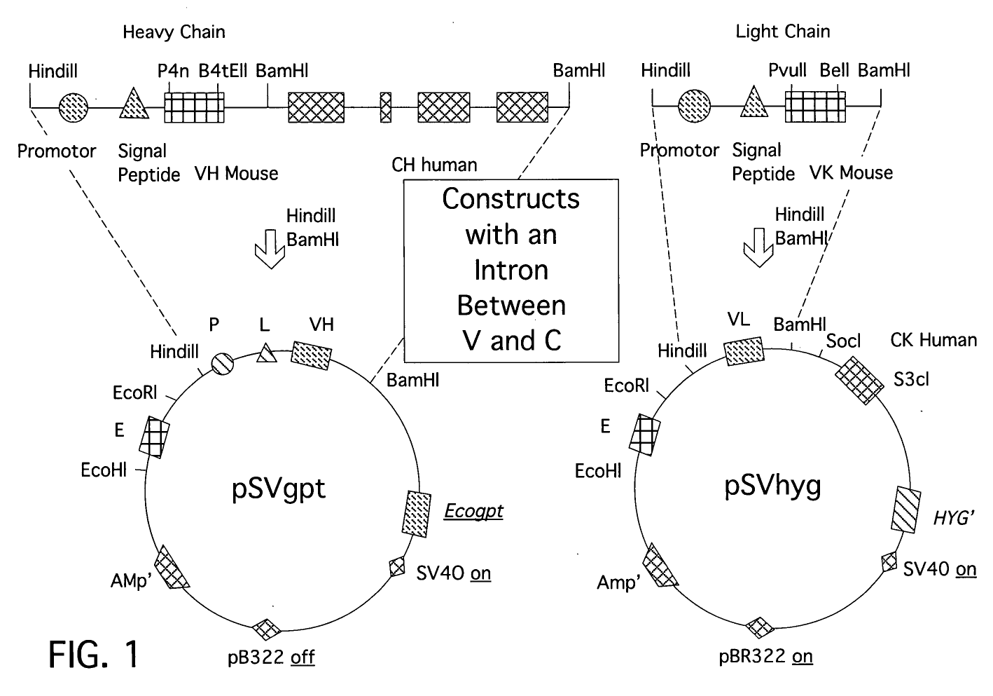

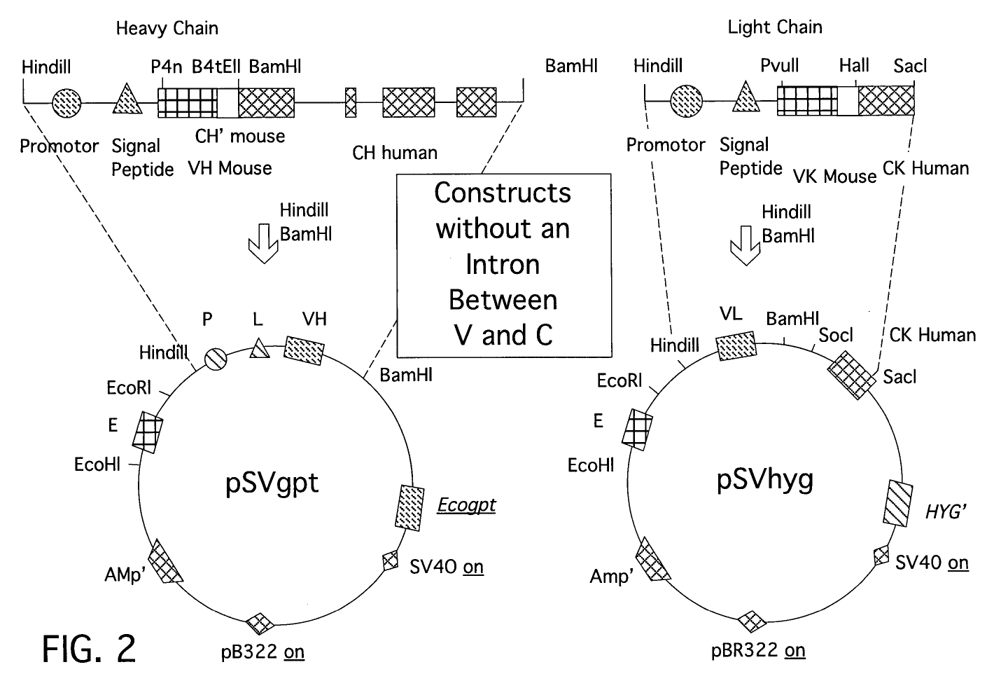

FIGS. 1 and 2 show schematically the construction of plasmids encoding the chimeric L and H-chain genes with and without an intron between the V- and C-regions.

3. Chimeric Heavy Chain with Intron (pSVgptGFH1)

The V_{H}-region of D9D10 obtained with PCR was inserted as a PstI-BstEII fragment into vector M13VHPCR1. This vector was described by Orlandi et al., Proc. Natl. Acad. Sci. USA 81:7161-7165 (1984). Thus, the phage M13VHIFNG was obtained. The (PR-L-V_{H})-region was subsequently inserted as a BamHI-HindIII-fragment into the expression vector pSVgptMoVHnp through which it was positioned behind the Ig-enhancer (E) of the expression vector. Thus, clone pSVgptVHIFNG Was obtained. The genomic human Γ-sequence of the vector pBGS18HuIgG1 (a gift from Dr. Honjo, Department of Medical Chemistry, Kioto, Japan) was inserted behind the PR-L-V_{H}-region in the unique BamHI-site of pSVgptVHIFNG to obtain the vector pSVgptGFH1 encoding a chimeric heavy chain with an intron.

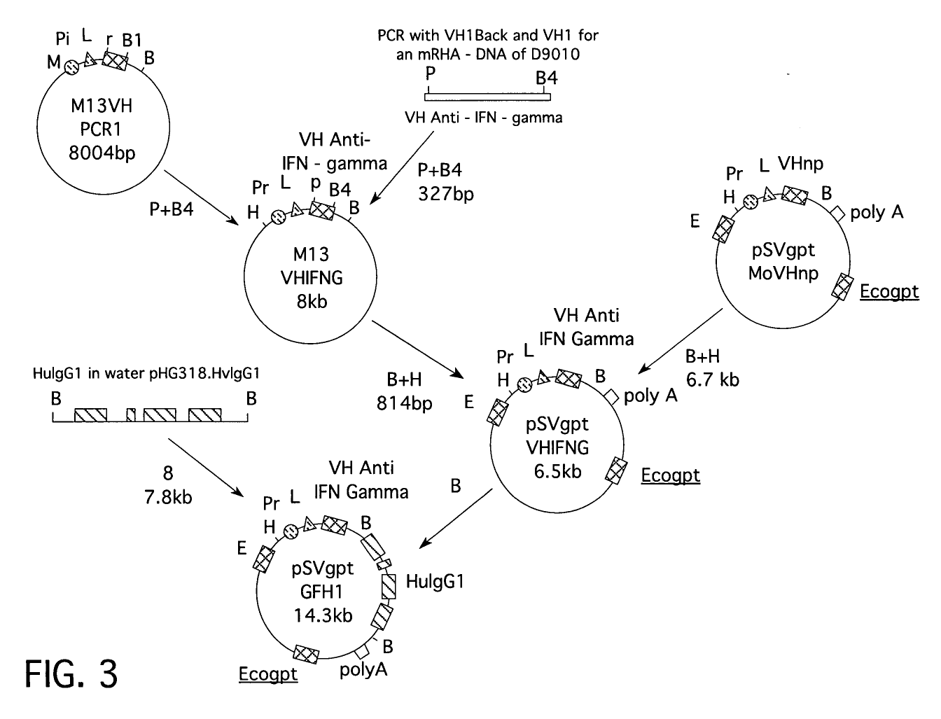

The construction of this chimeric heavy chain is schematically demonstrated in FIG. 3.

4. Chimeric Light Chain with an Intron (pSVhygGFL1)

The V_{K}-fragment of D9D10 was cloned as a PvuII-BglII-fragment into the PvuII-BclI-restriction site of vector M13VKPCR1. Since BclI only splices when the adenin residues in this site (TGATCA) are not methylated, damnegative E. coli cells had to be used to work with this phage. For practical reasons the (PR-L-V_{K})-region of M13VKPCR1 was first inserted into the vector p247 as a HindIII-BamHI fragment. Vector p247 is a pEG03 derived vector without the two PvuII sites. With the thus-obtained vector pEGVKPCR1 dam-negative E. coli cells (JM110-strain) were transformed. Subsequently, the HindIII-BamHI-fragment was, after insertion of the V_{K}-region of D9D10 (pEGVKIFNG), transferred to the expression vector pSVhygHuCK. Because the V_{K}-region comprises an internal BamHI-site in the FR2-region, two cloning steps were necessary. First, a 370 Bp HindII-BamHI-fragment was cloned into pSVhygHuCK. Subsequently, the remaining 240 bp BamHI-fragment was inserted into the unique BamHI-restriction site of this construct, thus obtaining an expression vector encoding a chimeric light chain with an intron between V and C (pSVhygGFL1).

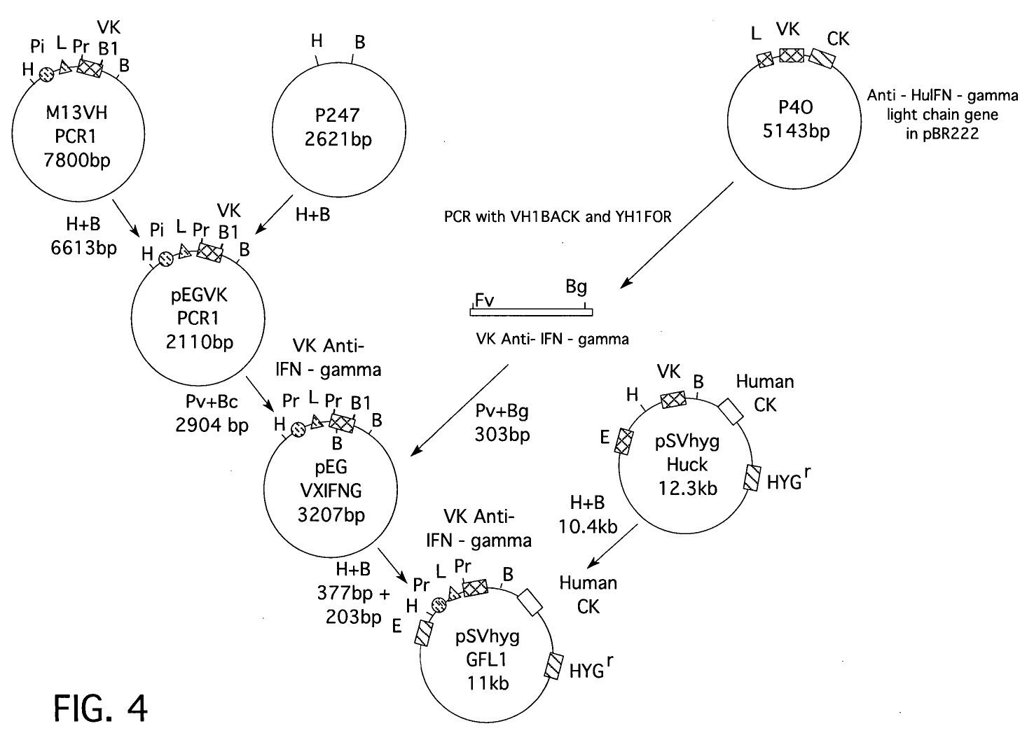

FIG. 4 shows schematically the construction of this expression vector.

5. Construction of Chimeric Heavy Chain

The human Γ1 region comprises a unique ApaI site at the beginning of the C-region. To use this site for linkage with the V_{H}-region, an ApaI-site had to be created in the beginning of the murine C-region. Therefore, the PCR-method was used.

The primers were as follows:

VH1BACK:

(SEQ ID NO: 1)

5′ AGGTSMARCTGCAGSAGTCWGG 3′
PstI
VH1APA:

(SEQ ID NO: 5)

5′ GATGGGCCCGTCGTTTTGGCTGAGGAG 3′
ApaI

wherein:
S = C or G
M = A or C
R = A or G
W = A or T

With 1 μg total RNA from D9D10 hybridoma cells, the first cDNA strand was synthesized with random hexanucleotides as primers. Amplification with VH1BACK and VH1APA yielded a 380 bp (V_{H}-C_{H′})-fragment. This fragment was eluted out of a 2% LMP agarose gel, digested with PstI and ApaI and cloned into vector pEG02, a pUC19-derived vector in which an ApaI site was inserted. Then the (V_{H}-C_{H′}) sequence was cloned in vector pEGVHPCR2 as a PstI-BamHI fragment to yield plasmid P351. The pEGVHPCR2-vector was obtained by the insertion of the HindIII-BamHI fragment of M13VHPCR1 into the plasmid pEG04, another pUC19-derived vector. The human Γ1 sequence available from vector pBGS18-HuIgG1 was cloned in vector P351 as an ApaI fragment. The final construct (pSVgptGFH2) with the chimeric heavy chain without intron between the V- and C-regions was finally obtained by inserting the BamHI-HindIII-fragment from pEGGFH2 into the expression vector pSVgptMoVHnp.

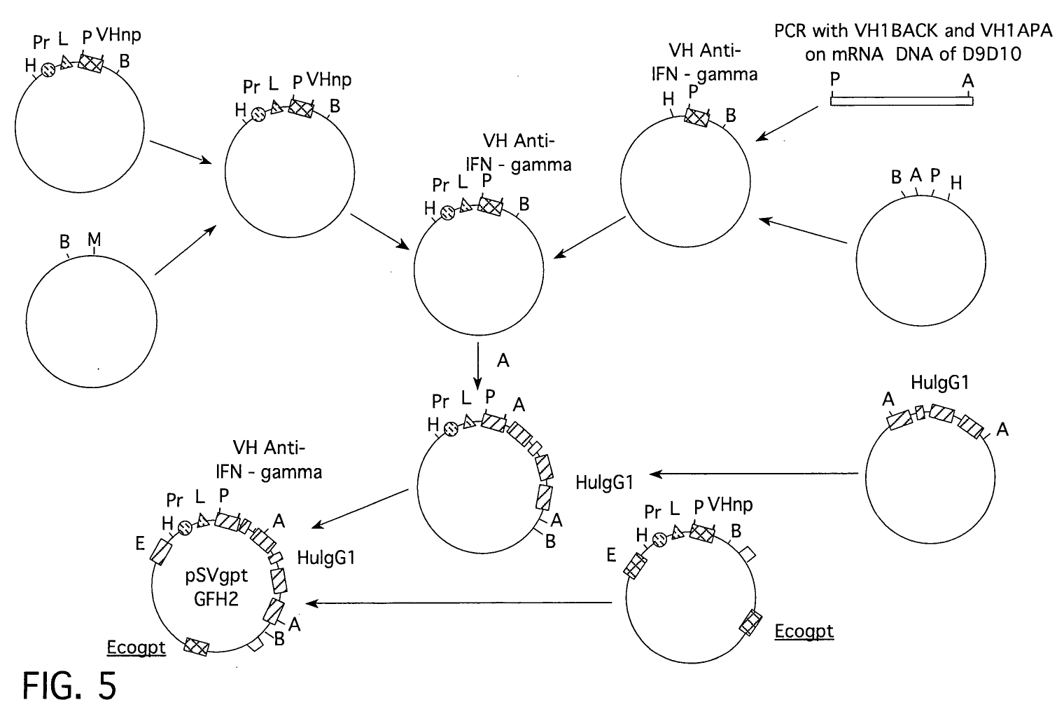

FIG. 5 shows schematically the construction strategy of the expression vector pSVgptGFH2.

6. Chimeric Light Chain without an Intron (pSVhygGFL2)

For the construction of a chimeric light chain without an intron between the variable and the constant kappa region, a unique NarI restriction site had to be created in the murine kappa chain gene. This was done by primer-mediated mutagenesis by means of PCR as described by Higuchi et al., Nucl. Acids Res. 16:7351-7367 (1988) and Ho et al., Gene 77:51-59 (1989). The PCR generates mutations by means of the primers. Therefore, the PCR has to be performed with two sets of primers. One of the primers of each set contains the desired mutation. Both mutated primers are complementary. The amplified DNAs show partial overlap because of the complementary primers. After amplification, the new sets of strands are denatured and renatured such that annealing takes place in the position of the complementary primers. Then another PCR is performed with only the non-mutated primers to fill in the missing parts of the strands. The first reaction was performed with the primers VK1BACK and VK1NAR, below. The second PCR used the primers CK1NAR and SSDNABACKW, below. Amplification took place by performing a PCR of 32 cycles for 35 seconds at 95° C., one minute at 60° C. and 45 seconds at 72° C. This yielded two overlapping primary PCR products that could hybridize after denaturation as a heteroduplex. A second amplification with only the two outermost primers (VK1BACK and SSDNABACKW) comprised 12 cycles with low annealing temperature (1 min. at 95° C., 1.3 min. at 45° C. and 35 sec. at 72° C.), followed by 20 cycles with higher annealing temperature (30 sec. at 55° C.). This yielded the total kappa chain sequence with a NarI site in the C-region.

The primers that were used were:

VK1BACK:

(SEQ ID NO: 6)

5′ GACATTCAGCTGACCCAGTCTCCA 3′
PvuII
VK1NAR:

(SEQ ID NO: 7)

5′ GATACAGTTGGCGCCGCATCAGCC 3′
NarI
CK1NAR:

(SEQ ID NO: 8)

5′ GCTGATGCGGCGCCAACTGT 3′
NarI
SSDNABACKW:

(SEQ ID NO: 9)

5′ AGCGGATAACAATTTCACACAGGA 3′

The human C_{K}-gene in the vector pUCHuCKnar already contained a NarI-site created by Dubois. The human C_{K}-region could then be isolated as a NarI-BamHI-fragment. The secondary PCR-fragment was digested with PvuII and NarI. The 320 bp (V_{K}-C_{K′}-fragment) was cloned into vector pEGVKPCR1 (digested with PvuII and BamHI) together with the NarI-BamHI-digested human C_{K}-region. Finally, the HindIII-SacI-fragment of pEGGFL2 was transferred to the expression vector pSVhygHuCK (digested with HindIII and SacI). This yielded pSVhygGFL2, the chimeric L-chain gene without intron between V and C.

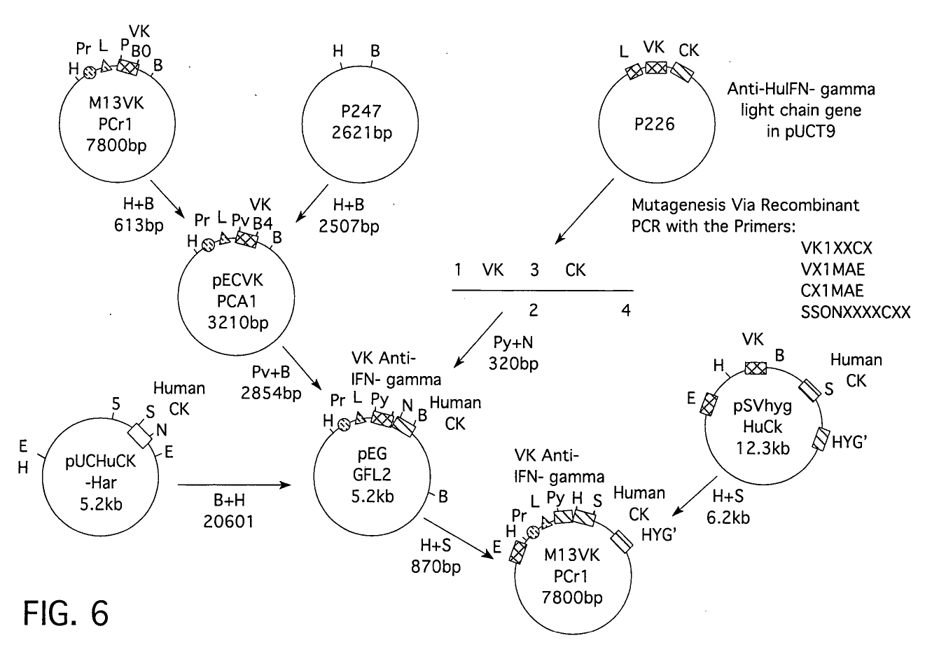

FIG. 6 shows schematically the construction of this expression vector.

### EXAMPLE 3

Transfection

Electroporation was performed using the method of Potter et al., Proc. Natl. Acad. Sci. USA 81:7161-7165 (1984). The cells were first washed in cold PBS, resuspended to 10⁶ cells/ml in PBS and kept on ice. 800 μl of this suspension was transferred into the cuvette of the electroporation device (0.4 cm Electrogene pulser; Bio-rad, California). The DNA was added hereto, and after 10 minutes incubation on ice, an electric shock of 200 volts at 960 μFD was applied. After a second incubation of 10 minutes on ice, the cells were centrifuged for 5 minutes at 1000 rpm and resuspended in 24 ml culture medium with 40 μg/ml gentamycin and divided into a 24 well plate (Nunc, Roskilde, Denmark). After an incubation of two days at 37° C., the medium was changed with selection medium. For the pSVgpt-vectors this was MEM with 5% dialysed FBS, 10 μg/ml thymidin, 250 μg/ml xanthin, 15 μg/ml hypoxanthin, 0.1 μg/ml (first change) or 0.5 μg/ml (all following changes) mycophenolic acid and 40 μg/ml gentamycin. For the pSVhyg vectors 400 μg/ml hygromycin B and 40μg/ml gentamycin were added to the medium.

After 10 days of incubation, the resistant clones could be distinguished. They were transferred to 96 well microtiter plates. As soon as the confluence was greater than 60%, the supematants were tested in an ELISA for antibody production. Positive clones were transferred to 80 cm² culture flasks.

### EXAMPLE 4

Expression of Chimeric Chains and Chimeric Antibodies

The chimeric light chain construct with an intron-between V and C, pSVhygGFL1, was transfected into 1.6×10⁶ Sp2/O—Ag14 cells (ATCC Accession Number CRL-1581; American Type Culture Collection, 12301 Parklawn Drive, Rockville, Md. 20852) by means of electroporation. After eight days in the presence of selection medium (400 μg/ml hygromycin), thirty wells showed resistant clones of which only 17 remained after three weeks of selection. The transfection efficiency, therefore, amounts to 0.9×10⁻⁵. These 17 clones were transferred to a microtiter plate, and when the confluence amounted to more than 50%, the supernatant was tested in an ELISA for the detection of the human kappa chain.

Seven clones (41%) had no detectable chimeric light chain product; five (29%) had a week, three (18%) a good, and two (12%) a very good production of the human kappa chain. The production and secretion of the complete chimeric antibody was showed with Western blot. Supematants of the human kappa producing 3B3- and 3D3 transfectomas were separated on a 15% SDS polyacrylamid gel. As a control, the supernatant of Sp2/O-cells was used and some dilutions of human serum. After blotting on a PVDF filter, the kappa chain was immunologically detected with the biotinylated goat-anti-human-kappa antibody. After coloring, it was seen that the light chains, produced by 3B3 and 3D2, ran as far as those of human serum. With the help of a molecular weight marker, the molecular weight was estimated at 29 to 30 kDa which is similar to that of a complete light chain. Clone 3D2 showed in the ELISA the highest chimeric L-chain production and was therefore chosen for the introduction of a chimeric H-chain gene. During a period of at least six months, no loss of chimeric L-chain production was determined.

The chimeric kappa-chain producing transfectoma 3D2 was used for the introduction of the chimeric heavy chain gene without intron between V and C.

Two separate electroporations were performed wherein 1×10⁶ 3D2 cells were transfected with 6 or 12 μg pSVgpt-GFH1. Transfected cells were spread out in a 24 well culture plate in MEM-medium. After two days, the cells were fed with Ecogpt selection medium with 0.15 μg/ml mycophenolic acid. Two days later the concentration was increased to 0.5 μg/ml. This was also the concentration for all further changes. Ten days after infection 72 wells showed resistant clones. The resistant clones were then transferred to a new 24 well culture plate. To allow the clones to grow faster, the cells were refreshed with MEM-medium (without mycophenolic acid) 14 days after transfection. Through this, 40 outgrowing clones could be isolated. This amounts to a final transfection efficiency of 1×10⁻⁵. When the confluence amounted to more than 50%, the supernatant was tested in the ELISA for the detection of the human Γ1-chain. These cells were also split and cultivated in double wherein one series was cultured in MEM-medium, while the other series was cultured in selection medium with 0.5 μg/ml mycophenolic acid.

### EXAMPLE 5

Construction of an Expression Vector for Fv-Fragments of Anti-HuIFN-Γ Antibody

For the construction of an expression vector for the Fv-fragments of anti-HuIFN-Γ the vector pSW1-VHD1.3-VKD1.3-TAG1 was used as described by Ward et al. (1989) Nature 341:544-546. The V_{H}-domain of the antibody D1.3 was eliminated out of the vector by digestion with PstI and BstEII and replaced by the V_{H}-fragment of an anti-human-interferon-Γ of the hybridoma cell line D9D10, according to formula Ia. The fragment was obtained in sufficient amounts as described in Example 5. The V_{K}-domain of the antibody D1.3 was then replaced by a SacI-XhoI-fragment according to Formula Ib comprising the V_{K}-region of anti-HuIFN-Γ. The expression vector thus obtained was named pSW1-Fv-anti-HuIFN-Γ.

The nucleotide sequences of the V_{H}- and V_{K}-regions, respectively (Formula Ia and Ib respectively) (SEQ ID NOS: 10 and 11, respectively) of the D9D10 antibody are represented in the accompanying formula form. The parts in bold represent the primers used in the PCR. The underlined parts represent the restriction sites.

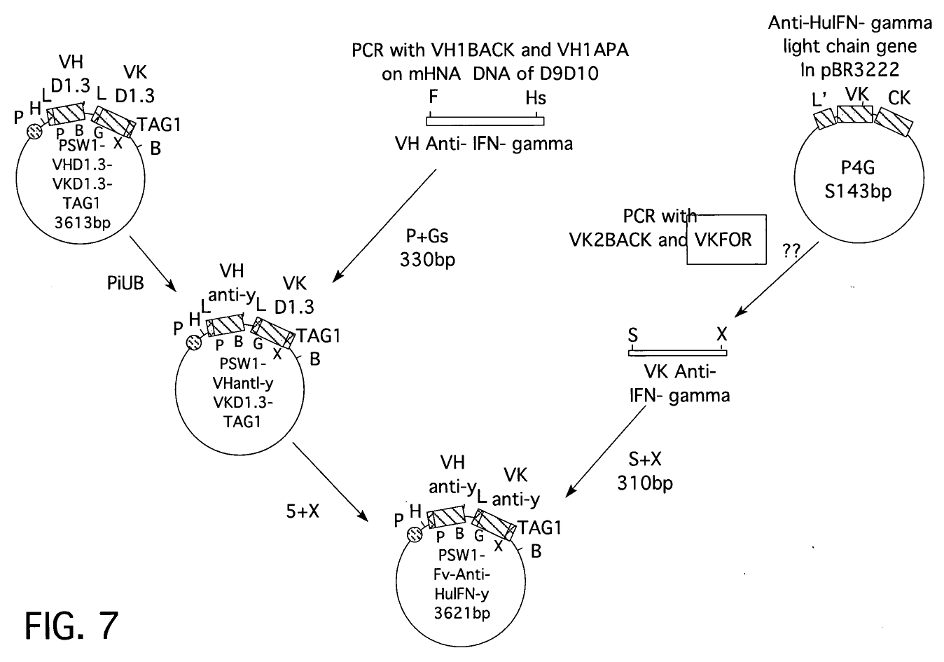

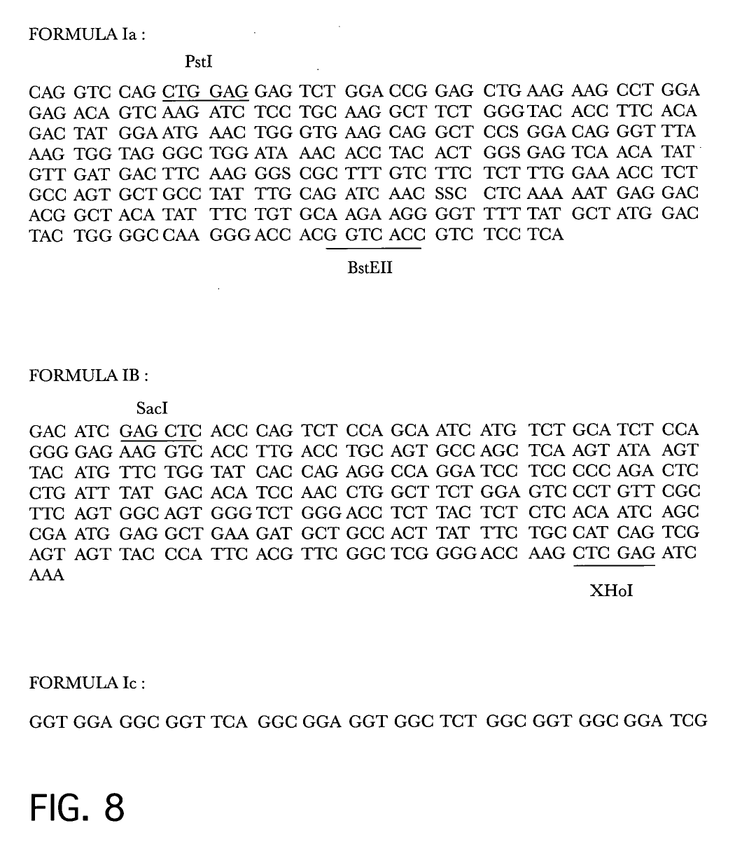

The strategy of construction is schematically represented in FIG. 7.

### EXAMPLE 6

Transformation of E. coli (DH5α)

Plasmid DNA was brought into E. coli cells according to the CaCl₂ method of Mandel et al. ((1970) J. Mol Biol. 53:159-162) except for the difference that only one heat shock was administered (instead of five), namely, 20 sec. at 37° C. for a 50 μl reaction, 45 sec. at 37° C. for a 100 μl reaction.

E. Coli DH5α cells (supE44, hsdR17(rₖ−, mₖ+), recA1, endA1, gyrA96, thi-1, relA1, del(argF-laczya)U169, ø80d-lacZdelM15) were spread out after transformation in LB-medium (1% Bacto-trypton); 0.5% yeast extract and 0.8% soft agar) on a solid LB-plate (1.5% agar) containing 100 μg/ml ampicillin, 0.02% X-gal en 0.25 mM IPTG. They were incubated overnight at 37° C. In this manner clone P330 was obtained.

### EXAMPLE 7

Expression of the Fv-Fragment of Anti-HuIFN-Γ

The bacterial clone comprising the expression vector for the production of the Fv of the anti-HuIFN-Γ-antibody was grown for 16 hours at 37° C. in LB-medium containing 100 μM ampicillin and 1% glucose. Glucose suppresses the lac-promotor which prevents the production of antibody fragments. Then the cells were washed twice in 50 mM NaCl and resuspended in LB-medium containing 100 μM ampicillin and 0.1-1 mM IPTG for induction of the promoter. This suspension was incubated for 2 to 20 hours in a shaking incubator at 37° C. The cells were then isolated by centrifugation (7000 rpm, 4° C.) and the supernatant tested in an ELISA (see Example 8) are stored at 4° C.

The periplasma was isolated as described by Skerra et al. (1988) Science 240: 1038-1041. After induction with IPTG, the cells were precipitated by centrifugation, resuspended in TES-buffer (0.2 M Tris-HCl pH 8.0; 0.5 mM EDTA; 0.5M sucrose; 10 ml/l culture) and subjected to an osmotic shock by addition of a 1/4 dilution of TES-buffer in water (15 ml/l culture). After 30 minutes on ice the suspension was centrifuged by 8000 rpm for 10 minutes at 4° C. The supernatant was centrifuged again at 20,000 rpm for 30 minutes to remove the remaining cell debris. The supernatant was directly tested, stored at 4° C. or frozen at −20° C.

### EXAMPLE 8

Detection of the Fv-Fragment of Anti-HuIFN-Γ

The binding of the Fv-fragment to the human IFN-Γ molecule was tested in an ELISA. The wells of a 96-well “MAXISORP NUNC-IMMUNO PLATE” were incubated overnight at 4° C. with 2 μg/ml recombinant HuIFN-Γ (Bioferon, Laupheim, Germany) in 100 μl of a 50 mM Tris-HCl pH 8.5 and 10 mM NaCl buffer. The stock solution of recombinant HuIFN-Γ was dissolved in 50 mM natrium phosphate and 100 mM ammonium acetate buffer with 12.5 mg/ml sucrose, to a concentration of 0.7 mg/ml pure protein.

The culture plate was decanted and to every well 250 μl blocker (PBS with 0.5% caseine and 0.01% merthiolate pH 7.5) was added. After incubation for one hour at 37° C. the cells were washed with 0.05% TWEEN-20 in PBS containing 0.01% merthiolate (washing buffer), after which the Fv-fragment samples were added (any dilutions in blocker). After shaking for 2 hours at 37° C., the material that was not bound was washed away and 100 μl of a 1 μg/ml anti-TAG1 antibody in blocker was added to every well. Subsequently, the plate was again shaken for two hours and washed three times. After that, a biotinylated sheep anti-mouse-Ig-antibody was added (Amersham, Buckinghamshire, GB, 100 μl of a 1/1000 dilution). After one hour shaking at 37° C. and three times washing, the reaction was finished with the biotinylated peroxidase-streptavidin complex, H₂O₂ and ABTS.

Because no anti-idiotype antibody was available, the detection was performed by means of the TAG1-polypeptide that was linked to the V_{K}-region.

The highest expression of Fv in the supernatant of 18 hours incubated cell culture amounted to 30 binding units. This means that a 1/30 dilution of the fluid was still positive (green color) in the ELISA for HuIFN-Γ binding. Almost no Fv was demonstrated in the periplasma for the same induction. This is caused by the fact that the Fv-concentration in the periplasma reaches its maximal value after an induction of two hours, after which the Fv leaks away into the culture liquid and after 18 hours is found substantially therein. This “leakage” out of the cells could be due to a stress situation in the bacteria caused by a high production of a strange protein. In the fluid of cells that were not induced (absence of IPTG), a basal amount of Fv was always present while the fluid of cells with a suppressed promoter, because of the presence of glucose, contained no detectable Fv-material.

The periplasma preparation method provided already a substantial enrichment of the produced Fv because the volume was much small in comparison to that of the supernatant. The periplasmic fraction of one liter culture amounted to 12 ml (83 times more concentrated) and of a 1 to 3 ml culture that amounted to 75 μl (13 to 40 times more concentrated). After a two hour induction with 0.1 mM IPTG (the combination giving the highest induction in the periplasma) the fluid as well as the periplasmic fraction were tested in the ELISA. The periplasma had a titer of 200 binding units; in the supernatant no Fv was detected.

From these experiments it is clear that E. coli, wherein the Fv-gen was introduced produced indeed Fv and that the Fv was capable of binding HuIFN-Γ. This means that neither the amino acid alterations in the 5′- and 3′-ends of the V-regions nor the linking of TAG1 to V_{K} have an important negative influence on the binding of the Fv of D9D10 to the HuIFN-Γ-molecule.

### EXAMPLE 9

Inhibition of the Antiviral Activity of HuIFN-Γ

To verify whether the Fv-fragment produced by E. coli can inhibit or neutralize the activity of HuIFN-Γ, in vitro neutralization tests were performed. For these A549-cells were used a human carcinoma cell line. This cell line was cultured in MEM with 10% “Newbom Calf Serum” (NCS) and is sensitive to the antiviral activity of HuIFN-Γ.

A human-IFN-Γ being just antivirally active (167 ng HuIFN-Γ with a specific activity of 2.8×10⁴U/μg on Wish cell; Biogen, Gent) were mixed with serial 1/2 logarithmic dilutions of the samples to be tested. These were incubated for 4 hours at 37° C. after which the mixtures were disinfected for 6 minutes under UV light. To all the wells 50,000 A549 cells were added and the whole was incubated for 20 hours in a CO₂-incubator until the cells had formed a monolayer. The infection was performed with a 1/300 dilution of the Encephalo Myocarditis Virus (EMCV). After 48 hours, when the virus control was totally affected, the living cells were colored with 100 μl of a neutral red solution (1/30 dilution neutral red in PBS with Ca²⁺ and Mg²⁺) and stored for 2 to 4 hours in the dark. Subsequently, they were washed with PBS (with Ca²⁺ and Mg²⁺) and 100 μl acidified alcohol was added to every well. After 20 minutes the plates were read in a Multiscan Titertek at 542 nm.

As controls, the samples in the absence of HuIFN-Γ and IFN-Γ in the absence of the sample were used. Cell and virus controls were also tested.

A 1/3 dilution of this fraction appeared to be capable to block the antiviral activity completely. When a combination of the 1/3 dilution together with a low IFN-Γ dose was brought on the cell, the EMC-virus could infect and kill the cells, contrary to the sample with the low IFN-Γ concentration alone, with which protection occurred. This inhibiting effect was not due to a possible toxicity of a component from the periplasma because in the absence of virus, the cells were not affected, neither to a stimulation of the virus growth, because the cells were not faster affected in the presence of the periplasmic fraction than in the absence thereof. In a 1/6 dilution of the periplasmic fraction, no inhibition of the antiviral activity was observed. The Fv-fragment that was purified on an affinity column showed the same neutralizing effects. This indicates that it is no other component from the periplasma that blocks the antiviral activity of HuIFN-Γ.

Moreover, periplasma of non-induced cells was not capable of neutralizing. This means that the Fv of the anti-HuIFN-Γ antibody of D9D10 is also capable of neutralizing the antiviral activity of HuIFN-Γ, despite minor changes in the 5′ and 3′-ends of the V-regions and the linking of TAG1 to V_{K}.

The above examples showed that the Fv-fragments have a neutralizing effect on the anti-viral activity of interferon-Γ. The absence of the constant domains in the Fv-fragments and the presence of human-derived constant domains in the chimeric antibodies will reduce the anti-immunoglobulin response when administered to patients.

The invention claimed is:

56. A method for producing biologically active Fv antibody fragments having a neutralizing effect on the anti-viral activity of human interferon-gamma (IFN-Γ) comprising:
a. inserting an isolated nucleic acid into a suitable expression vector, wherein the nucleic acid encodes an Fv antibody fragment which binds human IFN-Γ, said nucleic acid including nucleotide sequences encoding the V_{H} and V_{L} domains of the D9D10 monoclonal antibody and a nucleotide sequence encoding a linker peptide which links the V_{H} and V_{L} domains;
b. introducing the expression vector into a suitable host cell;
c. incubating the host cell to produce the Fv antibody fragment; and
d. isolating the Fv antibody fragment.

57. The method of claim 1, further comprising treating humans having a disease associated with IFN-Γ with the biologically active Fv antibody fragments, wherein the human disease is selected from the group consisting of endotoxic shock, local inflammation, cerebral malaria, and autoimmune arthritis.


SEQUENCE LISTING (9 sequences)

SEQ ID NO: 1 - DNA, 22 bp; SINGLE-stranded, UNKNOWN
aggtsmarctgcagsagtcwgg

SEQ ID NO: 2 - DNA, 32 bp; SINGLE-stranded, UNKNOWN
tgaggagacggtgaccgtggtcccttggcccc

SEQ ID NO: 3 - DNA, 24 bp; SINGLE-stranded, UNKNOWN
gacatcgagctcacccagtctcca

SEQ ID NO: 4 - DNA, 22 bp; SINGLE-stranded, UNKNOWN
gtttgatctcgagcttggtgcc

SEQ ID NO: 5 - DNA, 27 bp; SINGLE-stranded, UNKNOWN
gatgggcccgtcgttttggctgaggag

SEQ ID NO: 6 - DNA, 24 bp; SINGLE-stranded, UNKNOWN
gacattcagctgacccagtctcca

SEQ ID NO: 7 - DNA, 24 bp; SINGLE-stranded, UNKNOWN
gatacagttggcgccgcatcagcc

SEQ ID NO: 8 - DNA, 20 bp; SINGLE-stranded, UNKNOWN
gctgatgcggcgccaactgt

SEQ ID NO: 9 - DNA, 24 bp; SINGLE-stranded, UNKNOWN
agcggataacaatttcacacagga